(19) United States
(12) Patent Application Publication
(10) Pub. No.: US 2023/0174535 A1
(43) Pub. Date: Jun. 8, 2023

(54) INHIBITORS OF FIBROBLAST GROWTH FACTOR RECEPTOR KINASES

(71) Applicant: Kinnate Biopharma Inc., San Diego, CA (US)

(72) Inventors: Stephen W. KALDOR, San Diego, CA (US); John TYHONAS, San Diego, CA (US)

(21) Appl. No.: 17/605,127

(22) PCT Filed: May 14, 2020
(86) PCT No.: PCT/US2020/032939

§ 371 Date: Oct. 20, 2021

Related U.S. Application Data
(60) Provisional application No. 62/849,534, filed on May 17, 2019.
(60) Provisional application No. 62/907,491, filed on Sep. 27, 2019.

(51) Int. Cl. C07D 487/04

(52) U.S. Cl. CPC C07D 487/04

(57) ABSTRACT

Provided herein are inhibitors of fibroblast growth factor receptor kinases, pharmaceutical compositions comprising said compounds, and methods for using said compounds for the treatment of diseases.

### CROSS REFERENCE TO RELATED APPLICATIONS

This application claims the benefit of U.S. Provisional Application No. 62/907,491, filed Sep. 27, 2019, and U.S. Provisional Application No. 62/849,534, filed May 17, 2019, both of which are incorporated by reference in their entirety within the disclosure of this application.

### BACKGROUND

Fibroblast growth factor receptors (FGFRs) are a subfamily of receptor tyrosine kinases (RTKs) that bind to members of the fibroblast growth factor family of proteins. Deregulation of the fibroblast growth factor/FGF receptor network occurs frequently in tumors. Accordingly, therapies that target abberant FGFR kinase activity are desired for use in the treatment of cancer and other disorders.

### BRIEF SUMMARY OF THE INVENTION

Provided herein are inhibitors of fibroblast growth factor receptor (FGFR) kinases, pharmaceutical compositions comprising said compounds, and methods for using said compounds for the treatment of diseases.

One embodiment provides a compound, or pharmaceutically acceptable salt or solvate thereof, having the structure of Formula (I):

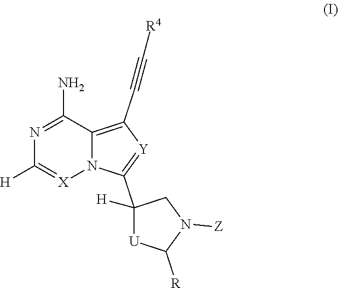

wherein,
X is C—H or N;
Y is C—H or N;
Z is selected from a group having the structure:

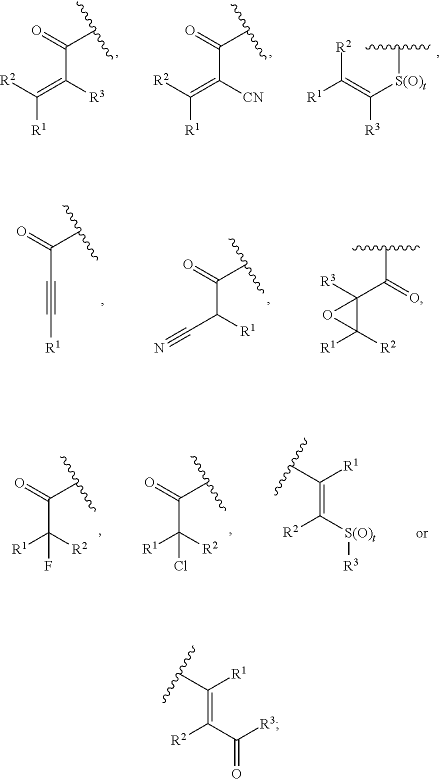

t is 1 or 2;
R¹, R², and R³ are each independently selected from hydrogen, optionally substituted C1-C4 alkyl, or optional substituted heterocyclylalkyl;
R⁴ is an optionally substituted C6 aryl;
U is —CH₂—, or a bond;
R is selected from optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂; and

each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

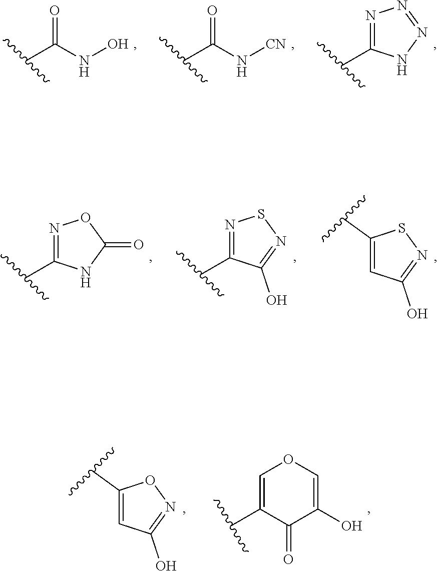

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

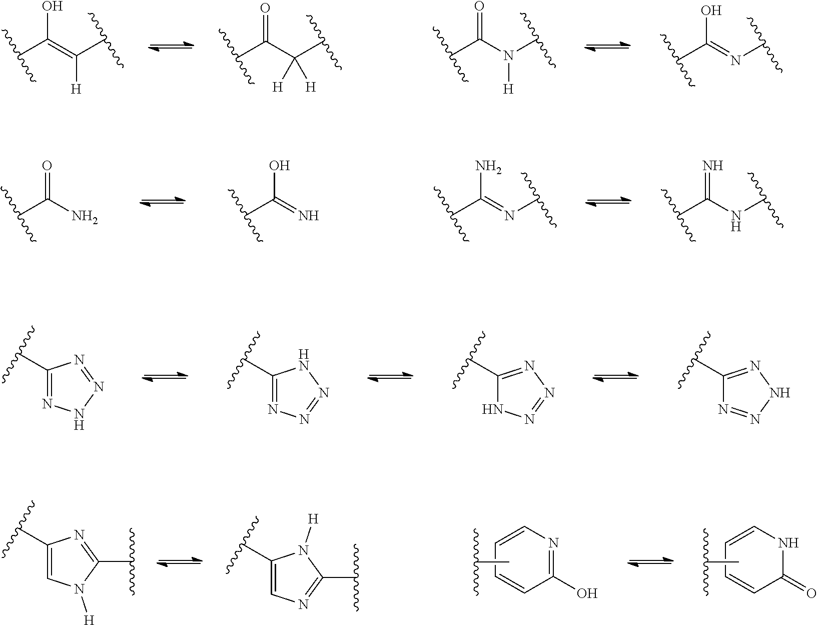

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Raj ender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

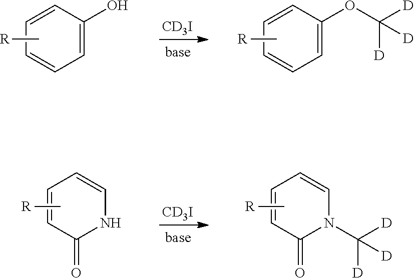

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

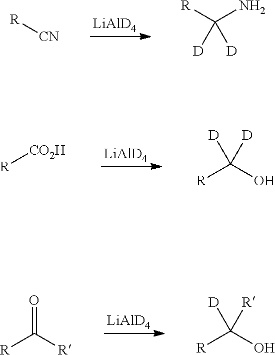

Deuterium gas and palladium catalyst are employed to reduce unsaturated carbon-carbon linkages and to perform a reductive substitution of aryl carbon-halogen bonds as illustrated, by way of example only, in the reaction schemes below.

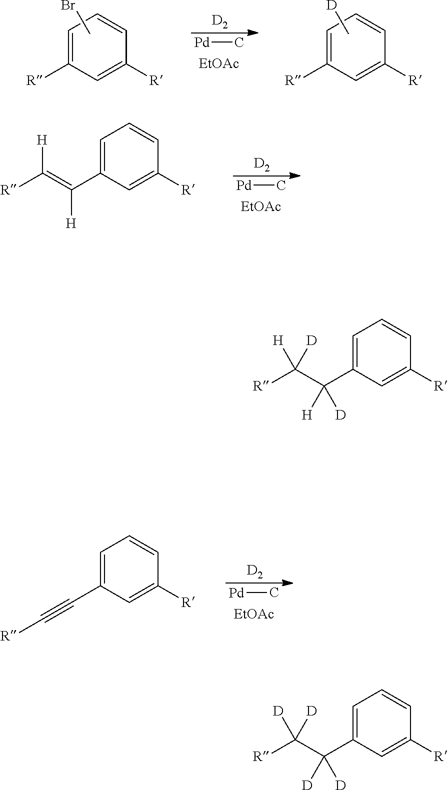

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the inhibitor of fibroblast growth factor receptors (FGFRs) compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

Fibroblast Growth Factor Receptor (FGFR)

Fibroblast growth factor receptors (FGFRs) are a subfamily of receptor tyrosine kinases (RTKs) that bind to members of the fibroblast growth factor family of proteins. FGFR genes generally contain 18 exons, possess similar exon-intron organization, and are randomly dispersed throughout the genome with no apparent linkages to FGF gene locations. FGFRs are differentially expressed in a tissue-specific manner throughout development and into adulthood and comprise an extracellular ligand-binding domain, a single-transmembrane domain, and a split intracellular kinase domain. The extracellular region contains two to three immunoglobulin (Ig)-like domains that are involved in FGF binding. These Ig-like domains regulate both ligand affinity and ligand specificity. The intracellular region has the functional domain responsible for FGFR tyrosine kinase activity, as well as additional sites that play a role in protein binding and phosphorylation or autophosphorylation of the receptor molecule. Fibroblast growth factor receptor pharmacology has been reviewed in the scientific literature by Porta et al. (Critical Reviews in Oncology/Hematology 113 (2017) 256-67) and Babina and Turner (Nature Review-Cancer 2017 doi: 10.1038/nrc.2017.8).

The FGFR family comprises of four family members—FGFR1, FGFR2, FGFR3, and FGFR4, but the four members are capable of producing multiple receptor isoforms through alternative splicing of primary transcripts. A closely-related receptor which lacks the FGF signaling tyrosine kinase domain, FGFR5, (also known as FGFRL1) was recently discovered on the basis of interaction with FGFR-binding ligands, known as fibroblast growth factors (FGFs) (Trueb B. Biology of FGFRL1, the fifth fibroblast growth factor receptor. Cell Mol Life Sci. 2011; 68(6):951-964). Collectively, FGFR signaling is associated with the activation of multiple cellular cascades and responses such as cell growth, proliferation, differentiation, and survival (Thisse B et al. Functions and regulations of fibroblast growth factor signaling during embryonic development. Dev Biol. 2005; 287(2):390-402; Wesche J et al. Fibroblast growth factors and their receptors in cancer. Biochem J. 2011; 437(2):199-213; Haugsten E M et al. Roles of fibroblast growth factor receptors in carcinogenesis. Mol Cancer Res. 2010; 8(11):1439-1452).

Numerous human pathological conditions are associated with the deregulation of FGFR signaling. Aberrant FGFR signaling is largely attributed to several underlying mechanisms involving gene amplification, gain-of-function coding mutation, gene fusions, single nucleotide polymorphism (SNP), ligand availability and impaired termination program in FGF-mediated signaling (Tiong K H et al. Functional roles of fibroblast growth factor receptors (FGFRs) signaling in human cancers. Apoptosis. 2013; 18(12):1447-68). In addition, a further layer of complexity is added by the fact that FGFRs are subjected to alternative splicing, giving rise to multiple isoforms which may promote or repress tumorigenesis, under different circumstances.

FGFR Fusions

FGFR fusions in human cancers are classified into type 1 fusions caused by chromosomal translocations in hematological malignancies, and type 2 fusions caused by chromosomal rearrangements in solid tumors (FGFR inhibitors: Effects on cancer cells, tumor microenvironment and whole-body homeostasis (Review). Int J Mol Med. 2016; 38(1):3-15). Both types of FGFR fusion proteins are endowed with oncogenic potential through the acquisition of protein-protein-interaction modules from fusion partners for ligand-independent dimerization and/or recruitment of aberrant substrates. Human FGFR fusion proteins generally consist of two main segments—the anterior being a dimerized domain from a partnering gene and tyrosine kinase domain at the posterior (Garcia-Closas M et al. Heterogeneity of breast cancer associations with five susceptibility loci by clinical and pathological characteristics. PLoS Genet. 2008; 4(4):e1000054). Unlike wild type receptors, mutant FGFRs are expressed intracellularly and retained in the cytosol, thus they escape the typical receptor degradation processes, further prolonging the activation signal.

Deregulation of the fibroblast growth factor (FGF)/FGF receptor (FGFR) network occurs frequently in tumors, resulting in the development of FGF/FGFR-targeting therapies as the focus of several basic, preclinical, and clinical studies.

Heteroaromatic FGFR Inhibitory Compounds

In one aspect, provided herein is a heteroaromatic FGFR inhibitory compound.

One embodiment provides a compound, or pharmaceutically acceptable salt or solvate thereof, having the structure of Formula (I):

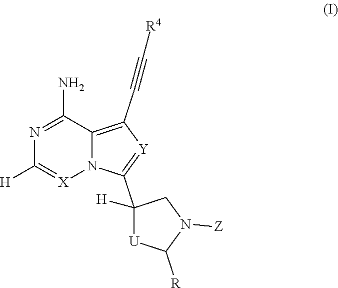

wherein,
X is C—H or N;
Y is C—H or N;
Z is selected from a group having the structure:

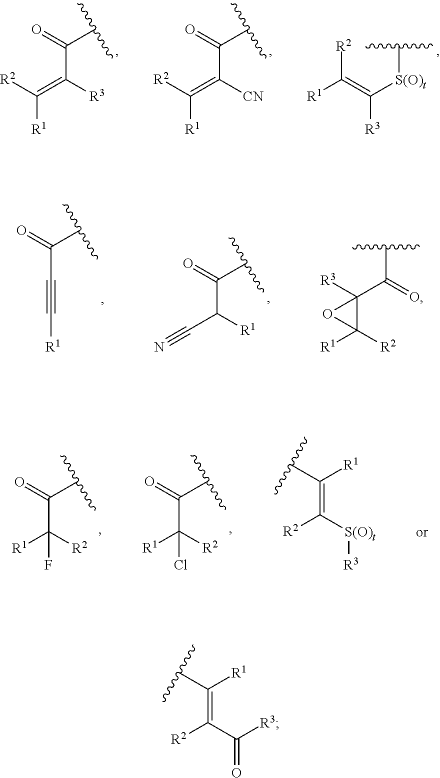

t is 1 or 2;
R¹, R², and R³ are each independently selected from hydrogen, optionally substituted C1-C4 alkyl, or optional substituted heterocyclylalkyl;
R⁴ is an optionally substituted C6 aryl;
U is —CH₂—, or a bond;
R is selected from optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂; and
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl.

Another embodiment provides the compound of Formula (I), or a pharmaceutically acceptable salt or solvate thereof, wherein X is C—H, and Y is C—H.

Another embodiment provides the compound of Formula (I), or a pharmaceutically acceptable salt or solvate thereof, wherein X is C—H, and Y is N.

Another embodiment provides the compound of Formula (I), or a pharmaceutically acceptable salt or solvate thereof, wherein X is N, and Y is C—H.

Another embodiment provides the compound of Formula (I), or a pharmaceutically acceptable salt or solvate thereof, wherein X is N, and Y is N.

Another embodiment provides the compound of Formula (I), or a pharmaceutically acceptable salt or solvate thereof, wherein Z is

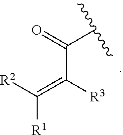

Another embodiment provides the compound, or pharmaceutically acceptable salt or solvate thereof, wherein R² is hydrogen. Another embodiment provides the compound, or pharmaceutically acceptable salt or solvate thereof, wherein R³ is hydrogen. Another embodiment provides the compound, or pharmaceutically acceptable salt or solvate thereof, wherein R² and R³ are hydrogen. Another embodiment provides the compound, or pharmaceutically acceptable salt or solvate thereof, wherein R¹ is hydrogen. Another embodiment provides the compound, or pharmaceutically acceptable salt or solvate thereof, wherein R¹ is optionally substituted C1-C4 alkyl. Another embodiment provides the compound, or pharmaceutically acceptable salt or solvate thereof, wherein R¹ is optionally substituted C1-C2 alkyl. Another embodiment provides the compound, or pharmaceutically acceptable salt or solvate thereof, wherein R¹ is optionally substituted C1 alkyl. Another embodiment provides the compound, or pharmaceutically acceptable salt or solvate thereof, wherein the optionally substituted alkyl is substituted with an optionally substituted amino group. Another embodiment provides the compound, or pharmaceutically acceptable salt or solvate thereof, wherein the optionally substituted amino group is a dimethylamino.

Another embodiment provides the compound of Formula (I), or a pharmaceutically acceptable salt or solvate thereof, wherein U is a bond.

Another embodiment provides the compound of Formula (I), or a pharmaceutically acceptable salt or solvate thereof, wherein U is —CH₂—.

Another embodiment provides the compound of Formula (I), or a pharmaceutically acceptable salt or solvate thereof, wherein R⁴ is an unsubstituted phenyl. Another embodiment provides the compound, or a pharmaceutically acceptable salt or solvate thereof, wherein R⁴ is a substituted phenyl. Another embodiment provides the compound, or a pharmaceutically acceptable salt or solvate thereof, wherein R⁴ is a substituted phenyl having a hydrogen at the 4-position. Another embodiment provides the compound, or a pharmaceutically acceptable salt or solvate thereof, wherein the substituted phenyl has 1 or 2 substituents. Another embodiment provides the compound, or a pharmaceutically acceptable salt or solvate thereof, wherein the substituted phenyl is 3,5-disubstituted. Another embodiment provides the compound, or a pharmaceutically acceptable salt or solvate thereof, wherein the substituted phenyl has 3 or 4 substituents. Another embodiment provides the compound, or a pharmaceutically acceptable salt or solvate thereof, wherein the substituted phenyl is 2,3,5,6-tetrasubstituted. Another embodiment provides the compound, or a pharmaceutically acceptable salt or solvate thereof, wherein the substituted phenyl is substituted with at least one substituent selected from halogen, —CN, optionally substituted C1-C4 alkyl, or optionally substituted C1-C3 alkoxy.

Another embodiment provides the compound of Formula (I), or a pharmaceutically acceptable salt or solvate thereof, wherein R is optionally substituted C1-C6 alkyl. Another embodiment provides the compound, or a pharmaceutically acceptable salt or solvate thereof, wherein R is optionally substituted C3-C7 carbocyclyl. Another embodiment provides the compound, or a pharmaceutically acceptable salt or solvate thereof, wherein R is optionally substituted C3-C7 carbocyclylalkyl. Another embodiment provides the compound, or a pharmaceutically acceptable salt or solvate thereof, wherein R is optionally substituted C3-C7 heterocyclyl. Another embodiment provides the compound, or a pharmaceutically acceptable salt or solvate thereof, wherein R is optionally substituted C3-C7 heterocyclylalkyl. Another embodiment provides the compound, or a pharmaceutically acceptable salt or solvate thereof, wherein R is optionally substituted C2-C7 alkenyl. Another embodiment provides the compound, or a pharmaceutically acceptable salt or solvate thereof, wherein R is —CO₂R⁵. Another embodiment provides the compound, or a pharmaceutically acceptable salt or solvate thereof, wherein R is —CONHR⁵. Another embodiment provides the compound, or a pharmaceutically acceptable salt or solvate thereof, wherein R is —CON(R⁵)₂.

In some embodiments, the heteroaromatic FGFR kinase inhibitory compound disclosed herein has a structure provided in Table 1.

TABLE 1

| Synthetic | | |
|---|---|---|
| Chemistry Example | Compound Structure | Compound Name |
| 1 and 2 | 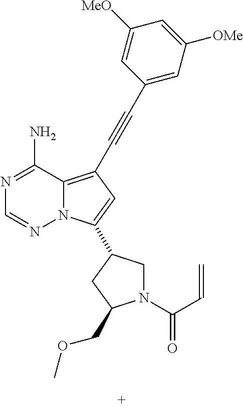 [SMILES: C=CC(=O)N1C[C@@H](c2cc(C#Cc3cc(OC)cc(OC)c3)c3c(N)ncnn23)C[C@@H]1COC] | 1-((2R,4S)-4-(4-amino-5-((3,5- dimethoxyphenyl)ethynyl)pyrrolo[2,1- f][1,2,4]triazin-7-yl)-2- (methoxymethyl)pyrrolidin-1-yl)prop- 2-en-1-one and 1-((2R,4R)-4-(4-amino-5-((3,5- dimethoxyphenyl)ethynyl)pyrrolo[2,1- f][1,2,4]triazin-7-yl)-2- (methoxymethyl)pyrrolidin-1-yl)prop- 2-en-1-one |
| | 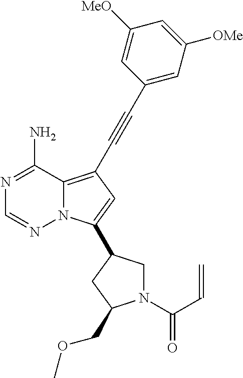 [SMILES: C=CC(=O)N1C[C@H](c2cc(C#Cc3cc(OC)cc(OC)c3)c3c(N)ncnn23)C[C@@H]1COC] | |
| 3 and 4 | 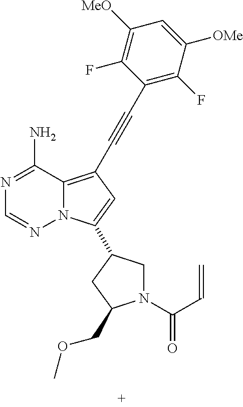 [SMILES: C=CC(=O)N1C[C@@H](c2cc(C#Cc3c(F)c(OC)cc(OC)c3F)c3c(N)ncnn23)C[C@@H]1COC] | 1-[(2R,4R)-4-[4-amino-5-[2-(2,6- difluoro-3,5- dimethoxyphenyl)ethynyl]pyrrolo[2,1- f][1,2,4]triazin-7-yl]-2- (methoxymethyl)pyrrolidin-1-yl]prop- 2-en-1-one and 1-[(2R,4S)-4-[4-amino-5-[2-(2,6- difluoro-3,5- dimethoxyphenyl)ethynyl]pyrrolo[2,1- f][1,2,4]triazin-7-yl]-2- (methoxymethyl)pyrrolidin-1-yl]prop- 2-en-1-one |
| | 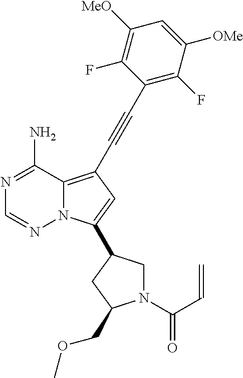 [SMILES: C=CC(=O)N1C[C@H](c2cc(C#Cc3c(F)c(OC)cc(OC)c3F)c3c(N)ncnn23)C[C@@H]1COC] | |
| 5 and 6 | 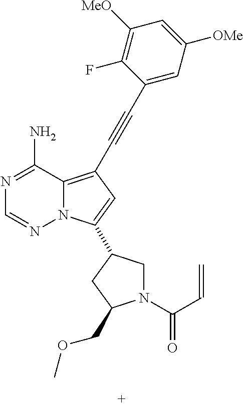 [SMILES: C=CC(=O)N1C[C@@H](c2cc(C#Cc3cc(OC)cc(OC)c3F)c3c(N)ncnn23)C[C@@H]1COC] | 1-[(2R,4R)-4-[4-amino-5-[2-(2-fluoro- 3,5-dimethoxyphenyl) ethynyl]pyrrolo[2,1-f][1,2,4]triazin-7- yl]-2-(methoxymethyl)pyrrolidin-1- yl]prop-2-en-1-one and 1-[(2R,4S)-4-[4-amino-5-[2-(2-fluoro- 3,5-dimethoxyphenyl) ethynyl]pyrrolo[2,1-f][1,2,4]triazin-7- yl]-2-(methoxymethyl)pyrrolidin-1- yl]prop-2-en-1-one |
| | 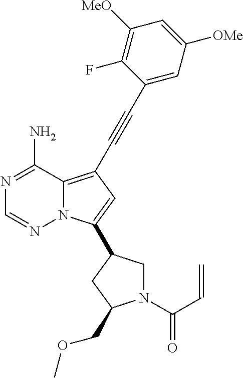 [SMILES: C=CC(=O)N1C[C@H](c2cc(C#Cc3cc(OC)cc(OC)c3F)c3c(N)ncnn23)C[C@@H]1COC] | |

In some embodiments, the heteroaromatic FGFR kinase inhibitory compound disclosed herein has a structure provided in Table 2A, wherein Z is selected from a substituent illustrated in Table 2B.

TABLE 2A

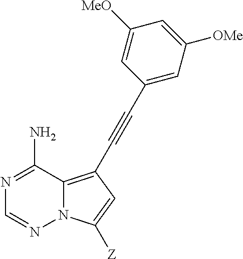

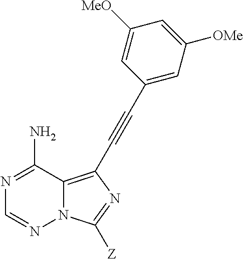

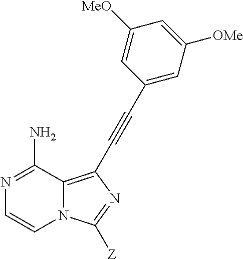

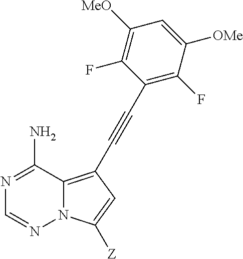

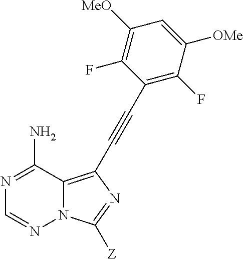

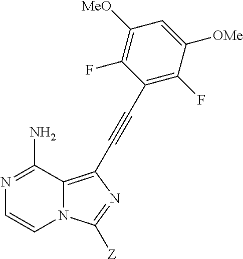

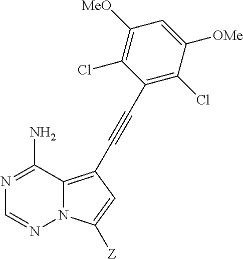

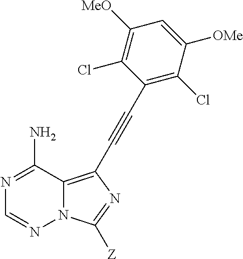

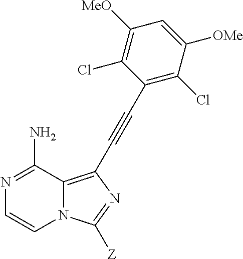

TABLE 2B

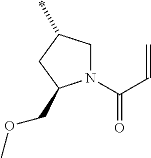 [SMILES: *[C@H]1C[C@H](COC)N(C(=O)C=C)C1]

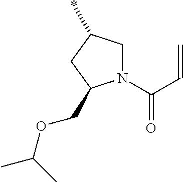 [SMILES: *[C@H]1C[C@H](COC(C)C)N(C(=O)C=C)C1]

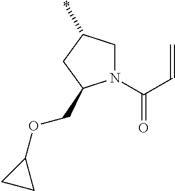 [SMILES: *[C@H]1C[C@H](COC2CC2)N(C(=O)C=C)C1]

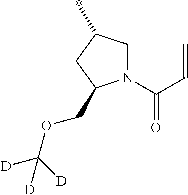 [SMILES: *[C@H]1C[C@H](COC([2H])([2H])[2H])N(C(=O)C=C)C1]

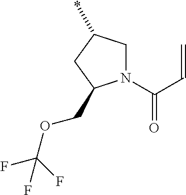 [SMILES: *[C@H]1C[C@H](COC(F)(F)F)N(C(=O)C=C)C1]

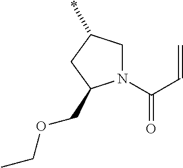 [SMILES: *[C@H]1C[C@H](COCC)N(C(=O)C=C)C1]

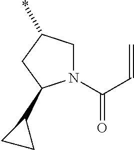 [SMILES: *[C@H]1C[C@H](C2CC2)N(C(=O)C=C)C1]

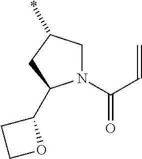 [SMILES: *[C@H]1C[C@H]([C@H]2CCO2)N(C(=O)C=C)C1]

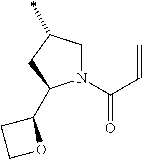 [SMILES: *[C@H]1C[C@H]([C@@H]2CCO2)N(C(=O)C=C)C1]

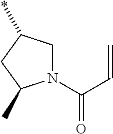 [SMILES: *[C@H]1C[C@H](C)N(C(=O)C=C)C1]

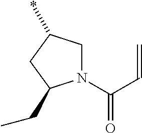 [SMILES: *[C@H]1C[C@H](CC)N(C(=O)C=C)C1]

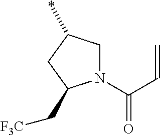 [SMILES: *[C@H]1C[C@H](CC(F)(F)F)N(C(=O)C=C)C1]

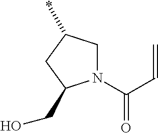 [SMILES: *[C@H]1C[C@H](CO)N(C(=O)C=C)C1]

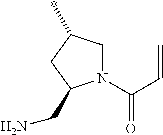 [SMILES: *[C@H]1C[C@H](CN)N(C(=O)C=C)C1]

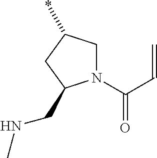 [SMILES: *[C@H]1C[C@H](CNC)N(C(=O)C=C)C1]

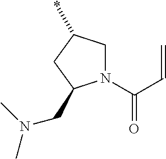 [SMILES: *[C@H]1C[C@H](CN(C)C)N(C(=O)C=C)C1]

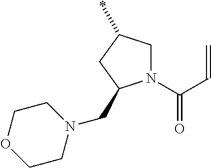 [SMILES: *[C@H]1C[C@H](CN2CCOCC2)N(C(=O)C=C)C1]

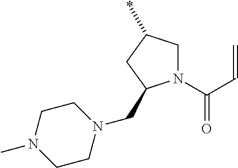 [SMILES: *[C@H]1C[C@H](CN2CCN(C)CC2)N(C(=O)C=C)C1]

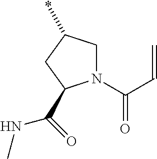 [SMILES: *[C@H]1C[C@H](C(=O)NC)N(C(=O)C=C)C1]

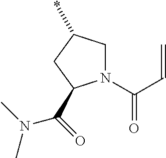 [SMILES: *[C@H]1C[C@H](C(=O)N(C)C)N(C(=O)C=C)C1]

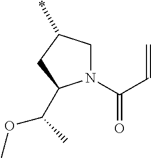 [SMILES: *[C@H]1C[C@H]([C@H](C)OC)N(C(=O)C=C)C1]

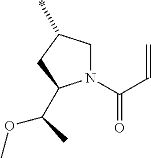 [SMILES: *[C@H]1C[C@H]([C@@H](C)OC)N(C(=O)C=C)C1]

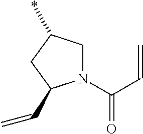 [SMILES: *[C@H]1C[C@H](C=C)N(C(=O)C=C)C1]

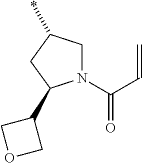 [SMILES: *[C@H]1C[C@H](C2COC2)N(C(=O)C=C)C1]

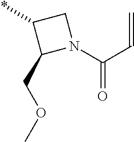 [SMILES: *[C@@H]1CN(C(=O)C=C)[C@H]1COC]

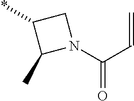 [SMILES: *[C@@H]1CN(C(=O)C=C)[C@H]1C]

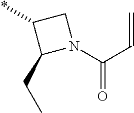 [SMILES: *[C@@H]1CN(C(=O)C=C)[C@H]1CC]

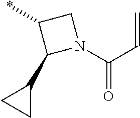 [SMILES: *[C@@H]1CN(C(=O)C=C)[C@H]1C1CC1]

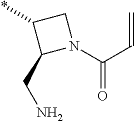 [SMILES: *[C@@H]1CN(C(=O)C=C)[C@H]1CN]

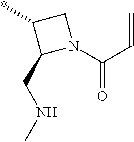 [SMILES: *[C@@H]1CN(C(=O)C=C)[C@H]1CNC]

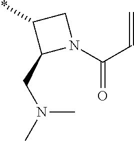 [SMILES: *[C@@H]1CN(C(=O)C=C)[C@H]1CN(C)C]

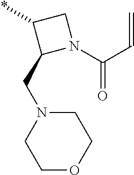 [SMILES: *[C@@H]1CN(C(=O)C=C)[C@H]1CN1CCOCC1]

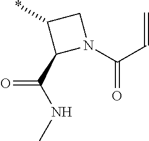 [SMILES: *[C@@H]1CN(C(=O)C=C)[C@H]1C(=O)NC]

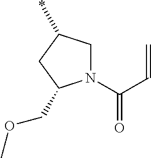 [SMILES: *[C@H]1C[C@@H](COC)N(C(=O)C=C)C1]

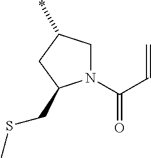 [SMILES: *[C@H]1C[C@H](CSC)N(C(=O)C=C)C1]

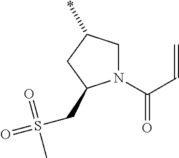 [SMILES: *[C@H]1C[C@H](CS(C)(=O)=O)N(C(=O)C=C)C1]

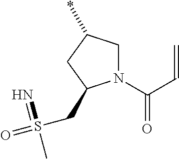 [SMILES: *[C@H]1C[C@H](CS(C)(=N)=O)N(C(=O)C=C)C1]

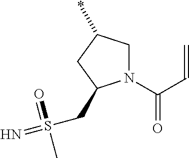 [SMILES: *[C@H]1C[C@H](CS(C)(=N)=O)N(C(=O)C=C)C1]

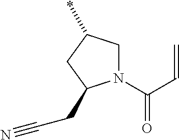 [SMILES: *[C@H]1C[C@H](CC#N)N(C(=O)C=C)C1]

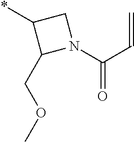 [SMILES: *C1CN(C(=O)C=C)C1COC]

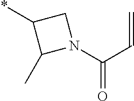 [SMILES: *C1CN(C(=O)C=C)C1C]

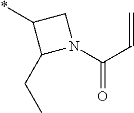 [SMILES: *C1CN(C(=O)C=C)C1CC]

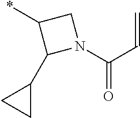 [SMILES: *C1CN(C(=O)C=C)C1C1CC1]

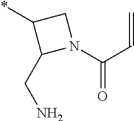 [SMILES: *C1CN(C(=O)C=C)C1CN]

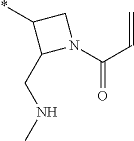 [SMILES: *C1CN(C(=O)C=C)C1CNC]

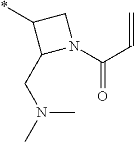 [SMILES: *C1CN(C(=O)C=C)C1CN(C)C]

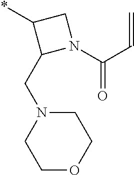 [SMILES: *C1CN(C(=O)C=C)C1CN1CCOCC1]

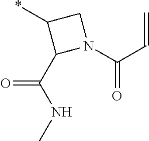 [SMILES: *C1CN(C(=O)C=C)C1C(=O)NC]

Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

Pharmaceutical Compositions

In certain embodiments, the heteroaromatic FGFR kinase inhibitory compound described herein is administered as a pure chemical. In other embodiments, the heteroaromatic FGFR kinase inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising at least one heteroaromatic FGFR kinase inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the heteroaromatic FGFR kinase inhibitory compound as described by Formula (I), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

In some embodiments, the heteroaromatic FGFR kinase inhibitory compound as described by Formula (I), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one heteroaromatic FGFR kinase inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (I), or a pharmaceutically acceptable salt or solvate thereof. In some embodiments, described herein is a method of treating cancer in a patient in need thereof comprising administering to the patient a pharmaceutical composition comprising a compound of Formula (I), or a pharmaceutically acceptable salt or solvate thereof, and a pharmaceutically acceptable excipient. In some embodiments, the cancer is breast cancer, colorectal cancer, ovarian cancer, pancreatic cancer, prostate cancer, or lung cancer.

Provided herein is the method wherein the pharmaceutical composition is administered orally.

Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### EXAMPLES

I. Chemical Synthesis

In some embodiments, the heteroaromatic FGFR kinase inhibitory compounds disclosed herein are synthesized according to the following examples. As used below, and throughout the description of the invention, the following abbreviations, unless otherwise indicated, shall be understood to have the following meanings:
° C. degrees Celsius
δ_{H} chemical shift in parts per million downfield from tetramethylsilane
DCM dichloromethane (CH₂Cl₂)
DMF dimethylformamide
DMSO dimethylsulfoxide
EA ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
T temperature
TFA trifluoroacetic acid
THF tetrahydrofuran

### Example 1 and 2: 1-((2R,4S)-4-(4-amino-5-((3,5-dimethoxyphenyl)ethynyl)pyrrolo[2,1-f][1,2,4]triazin-7-yl)-2-(methoxymethyl)pyrrolidin-1-yl)prop-2-en-1-one

### And

### 1-((2R,4R)-4-(4-amino-5-((3,5-dimethoxyphenyl)ethynyl)pyrrolo[2,1-f][1,2,4]triazin-7-yl)-2-(methoxymethyl)pyrrolidin-1-yl)prop-2-en-1-one

### Preparation 1: 1-(Tert-butyl) 2-methyl (2R,4R)-4-((tert-butyldiphenylsilyl)oxy)pyrrolidine-1,2-dicarboxylate

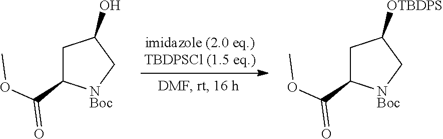

To a stirred solution of 1-(tert-butyl) 2-methyl (2R,4R)-4-hydroxypyrrolidine-1,2-dicarboxylate (8.00 g, 32.62 mmol) and imidazole (4.44 g, 65.23 mmol) in DMF (80 mL) was added tert-butyl(chloro)diphenylsilane (13.45 g, 48.93 mmol) at 0° C. over 0.5 hours. The reaction mixture was stirred for 16 h at room temperature. The resulting mixture was diluted with water (400 mL) and extracted with EA (3×300 ml). The combined organic layers was washed with brine (5×500 ml), dried over anhydrous Na₂SO₄ and filtered. The filtrate was concentrated under reduced pressure. The residue was purified by silica gel column chromatography, eluted with PE/EtOAc (6:1). The fractions contained desired product were combined and concentrated to afford 1-(tert-butyl) 2-methyl (2R,4R)-4-((tert-butyldiphenylsilyl)oxy)pyrrolidine-1,2-dicarboxylate (14.2 g, 90%) as a colorless crude oil. MS ESI calculated for C₂₇H₃₇NO₅Si [M+H]⁺, 484.24, found 484.25.

### Preparation 2: Tert-butyl (2R,4R)-4-[(tert-butyldiphenylsilyl)oxy]-2-(hydroxymethyl)pyrrolidine-1-carboxylate

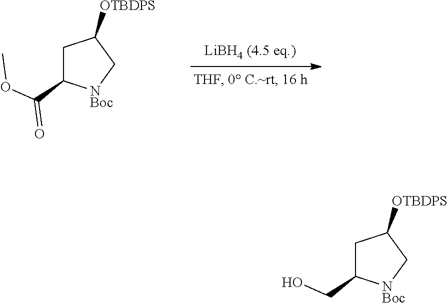

To a stirred solution of 1-(tert-butyl) 2-methyl (2R,4R)-4-((tert-butyldiphenylsilyl)oxy)pyrrolidine-1,2-dicarboxylate (30.00 g, 62.02 mmol) in THF (300 mL) was added LiBH₄ (6.08 g, 279.10 mmol) in portions at 0° C. under nitrogen atmosphere. The reaction mixture was stirred for 16 h at room temperature under nitrogen atmosphere. The resulting mixture was acidified to pH 5 with HCl (1M) at 0° C. and then basified to pH 8 with saturated NaHCO₃ (aq.). The resulting mixture was extracted with EtOAc (4×500 mL). The combined organic layers were dried over anhydrous Na₂SO₄ and filtered. The filtrate was concentrated under reduced pressure. The residue was purified by silica gel column chromatography, eluted with PE/EtOAc (5:1). The fractions contained desired product were combined and concentrated to afford tert-butyl (2R,4R)-4-[(tert-butyldiphenylsilyl)oxy]-2-(hydroxymethyl)pyrrolidine-1-carboxylate (24 g, 85%) as a light yellow oil. MS ESI calculated for C₂₆H₃₇NO₄Si [M+H]⁺, 456.25, found 456.30.

### Preparation 3: Tert-butyl (2R,4R)-4-[(tert-butyldiphenylsilyl)oxy]-2-(methoxymethyl)pyrrolidine-1-carboxylate

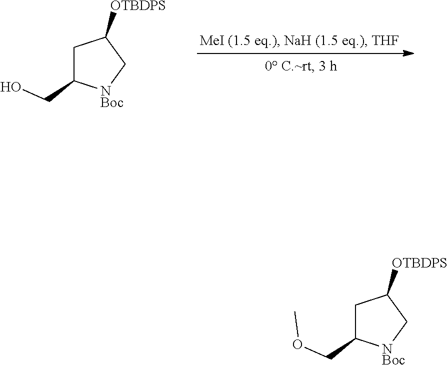

To a suspension of NaH (0.20 g, 8.33 mmol) in THF (18 ml) was added a solution of tert-butyl (2R,4R)-4-[(tert-butyldiphenylsilyl)oxy]-2-(hydroxymethyl)pyrrolidine-1-carboxylate (2.50 g, 5.49 mmol) in THF (64 ml) slowly at 0° C. under nitrogen atmosphere. After stirred at 0° C. for 1 hour, to the above mixture was added CH₃I (1.17 g, 8.23 mmol) dropwise at 0° C. The reaction mixture was stirred for additional 3 h at room temperature. The resulting mixture was diluted with water (60 mL), and then extracted with EA (30 ml×3). The combined organic layers was dried over anhydrous Na₂SO₄ and filtered. The filtrate was concentrated under reduced pressure to afford tert-butyl (2R,4R)-4-[(tert-butyldiphenylsilyl)oxy]-2-(methoxymethyl)pyrrolidine carboxylate (2.3 g, 89%) as a light yellow solid. MS ESI calculated for C₂₇H₃₉NO₄Si [M+H]⁺, 470.26, found 470.30.

### Preparation 4: Tert-butyl (2R,4R)-4-hydroxy-2-(methoxymethyl)pyrrolidine-1-carboxylate

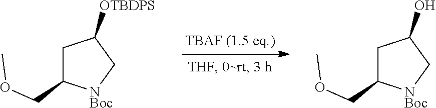

To a stirred solution of tert-butyl (2R,4R)-4-[(tert-butyldiphenylsilyl)oxy]-2-(methoxymethyl)pyrrolidine-1-carboxylate (46.30 g, 98.57 mmol) in THF (375 mL) was added tetra-n-butylammonium fluoride (1M in THF) (147.86 mL, 147.86 mmol) at 0° C. The reaction mixture was stirred at room temperature for 3 h. The resulting mixture was diluted with water (1 L) and extracted with EtOAc (3×500 mL). The combined organic layers was dried over anhydrous Na₂SO₄ and filtered. The filtrate was concentrated under reduced pressure. The residue was purified by silica gel column chromatography, eluted with PE/EtOAc (3:1). The fractions contained desired product were combined and concentrated to afford tert-butyl (2R,4R)-4-hydroxy-2-(methoxymethyl)pyrrolidine-1-carboxylate (16.6 g, 73%) as a light yellow oil. MS ESI calculated for C₁₁H₂₁NO₄ [M+H]⁺ 232.15, found 232.20.

### Preparation 5: Tert-butyl (2R,4S)-4-bromo-2-(methoxymethyl)pyrrolidine-1-carboxylate

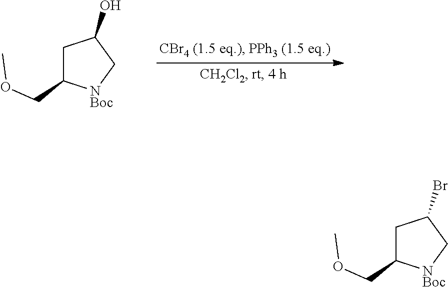 [SMILES: COC[C@H]1C[C@@H](O)CN1C(=O)OC(C)(C)C ; COC[C@H]1C[C@H](Br)CN1C(=O)OC(C)(C)C]

To a solution of tert-butyl (2R,4R)-4-hydroxy-2-(methoxymethyl)pyrrolidine-1-carboxylate (0.80 g, 3.46 mmol), CBr₄ (1.72 g, 5.19 mmol) in CH₂Cl₂ (8.00 mL) was added PPh₃ (1.36 g, 5.18 mmol) in portions over 30 min at 0-5° C. The reaction mixture was stirred for 4 h at room temperature under nitrogen atmosphere. To the above mixture EtOH (8.00 mL) was added. After stirred for additional 2 hours at room temperature, Et₂O (20.00 mL) was added until the precipitation was formed. The resulting mixture was stirred overnight. The resulting mixture was filtered and the filtrate was concentrated under reduced pressure. The residue was purified by silica gel column chromatography, eluted with 5% ethyl acetate in hexane. The fractions contained desired product were combined and concentrated to afford tert-butyl (2R,4S) bromo-2-(methoxymethyl)pyrrolidine-1-carboxylate (0.49 g, 48%) as a light yellow liquid. MS ESI calculated for C₁₁H₂₀BrNO₃ [M+H−tBu]⁺, 238.00, 240.00; found 237.85, 239.85.

### Preparation 6: Tert-butyl (2R)-4-[4-[bis(tert-butoxycarbonyl)amino]pyrrolo[2,1-f][1,2,4]triazin-7-yl]-2-(methoxymethyl)pyrrolidine-1-carboxylate

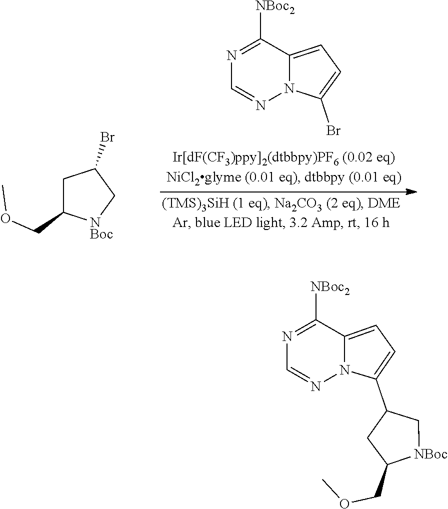

To a stirred solution of tert-butyl N-[7-bromopyrrolo[2,1-f][1,2,4]triazin-4-yl]-N-(tert-butoxycarbonyl)carbamate (0.29 g, 0.70 mmol), tert-butyl (2R,4S)-4-bromo-2-(methoxymethyl)pyrrolidine-1-carboxylate (0.41 g, 1.40 mmol), 2,6-dimethylpyridine (0.15 g, 1.40 mmol), tris(trimethylsilyl)silane (0.35 g, 1.40 mmol) and Ir[dF(CF₃)ppy]₂(dtbbpy)PF₆ (15.75 mg, 0.014 mmol) in DME (6.00 mL) were added 1,2-dimethoxyethane dihydrochloride nickel (1.54 mg, 0.007 mmol) and 4-tert-butyl-2-(4-tert-butylpyridin-2-yl)pyridine (1.88 mg, 0.007 mmol) at room temperature. The reaction mixture was degassed with argon for three times and stirred for 16 at ambient temperature under blue LED light. The resulting mixture was concentrated under reduced pressure. The residue was purified by silica gel column chromatography, eluted with 25% EA in PE. The fractions contained desired product were combined and concentrated to afford tert-butyl(2R)-4-[4-[bis(tert-butoxycarbonyl)amino]pyrrolo[2,1-f][1,2,4]triazin-7-yl]-2-(methoxymethyl)pyrrolidine-1-carboxylate (0.18 g, 47%) as a yellow oil. MS ESI calculated for C₂₇H₄₁N₅O₇ [M+H]⁺, 548.30, found 548.29.

### Preparation 7: Tert-butyl (2R)-4-[4-[(tert-butoxycarbonyl)amino]-5-iodopyrrolo[2,1-f][1,2,4]triazin-7-yl]-2-(methoxymethyl)pyrrolidine-1-carboxylate

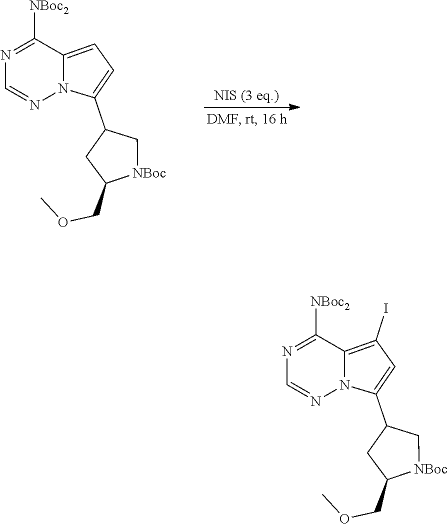

To a solution of tert-butyl (2R)-4-[4-[bis(tert-butoxycarbonyl)amino]pyrrolo[2,1-f][1,2,4]triazin-7-yl]-2-(methoxymethyl)pyrrolidine-1-carboxylate (0.10 g, 0.183 mmol) in DMF (2.00 mL) was added NIS (0.12 g, 0.548 mmol) was stirred for 16 h at room temperature. The resulting mixture was diluted with water (20 mL) and extracted with EA (3×10 mL). The combined organic layers was washed with brine (4×15 mL), dried over anhydrous Na₂SO₄ and filtered. The filtrate was concentrated under reduced pressure. The residue was purified by silica gel column chromatography, eluted with PE/EA (20/1˜3/1). The fractions contained desired product were combined and concentrated to afford tert-butyl (2R)-4-[4-[(tert-butoxycarbonyl)amino]-5-iodopyrrolo[2,1-f][1,2,4]triazin-7-yl]-2-(methoxymethyl)pyrrolidine-1-carboxylate (0.1 g, 95%) as a yellow oil. MS ESI calculated for C₂₇H₄₀IN₅O₇ [M+H]⁺, 674.20, found 674.20.

### Preparation 8: Tert-butyl (2R)-4-[4-amino-5-[2-(3,5-dimethoxyphenyl)ethynyl]pyrrolo[2,1-f][1,2,4]triazin-7-yl]-2-(methoxymethyl)pyrrolidine-1-carboxylate

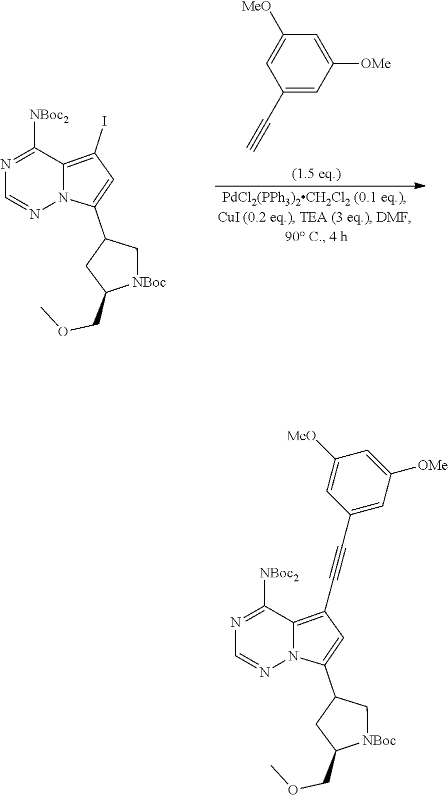

To a solution of tert-butyl (2R)-4-[4-[(tert-butoxycarbonyl)amino]-5-iodopyrrolo[2,1-f][1,2,4]triazin-7-yl]-2-(methoxymethyl)pyrrolidine-1-carboxylate (0.10 g, 0.174 mmol), 1-ethynyl-3,5-dimethoxybenzene (42.43 mg, 0.262 mmol), CuI (6.64 mg, 0.035 mmol) and Pd(dppf)Cl₂.CH₂Cl₂ (14.24 mg, 0.017 mmol) in DMF (2.00 mL) was added TEA (52.94 mg, 0.523 mmol) at room temperature under atmosphere. The reaction mixture was degassed with nitrogen for three times and stirred for 4 h at 90° C. The resulting mixture was diluted with water (20 mL) and extracted with EtOAc (3×10 mL). The combined organic layers was washed with brine (3×10 mL), dried over anhydrous Na₂SO₄ and filtered. The filtrate was concentrated under reduced pressure. The residue was purified by silica gel column chromatography, eluted with PE/EtOAc (0%-50%). The fractions contained desired product were combined and concentrated to afford tert-butyl (2R)-4-[4-amino-5-[2-(3,5-dimethoxyphenyl)ethynyl]pyrrolo[2,1-f][1,2,4]triazin-7-yl]-2-(methoxymethyl)pyrrolidine-1-carboxylate (46 mg, 49%) as a light yellow oil. MS ESI calculated for C₃₇H₄₉N₅O₉ [M+H−2 Boc]⁺, 508.35, found 508.40.

### Preparation 9: 5-((3,5-dimethoxyphenyl)ethynyl)-7-((5R)-5-(methoxymethyl)pyrrolidin yl)pyrrolo[2,1-f][1,2,4]triazin-4-amine

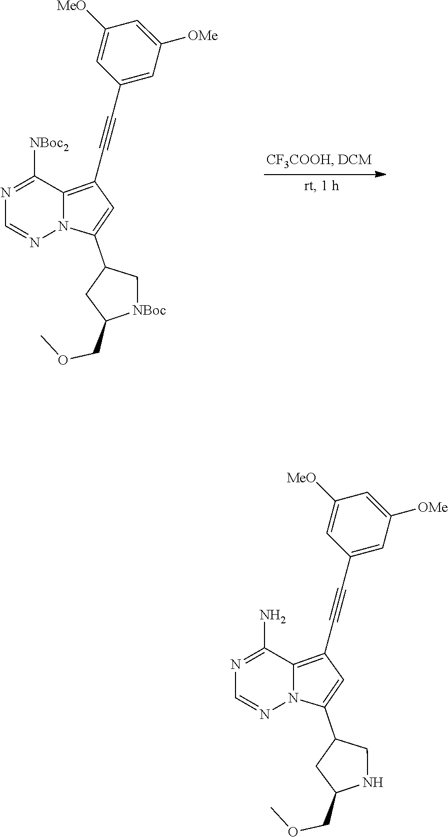

To a solution of tert-butyl (2R)-4-[4-amino-5-[2-(3,5-dimethoxyphenyl)ethynyl]pyrrolo[2,1-f][1,2,4]triazin-7-yl]-2-(methoxymethyl)pyrrolidine-1-carboxylate (46.00 mg, 0.091 mmol, 1.00 equiv) in DCM (4.00 mL) was added trifluoroacetic acid (1 mL) at room temperature. The reaction mixture was stirred for 1 h at room temperature. The resulting mixture concentrated under reduced pressure. The residue was basified to pH 8 with saturated NaHCO₃ (aq.). The resulting mixture was extracted with EtOAc (3×10 mL). The combined organic layers was washed with brine (2×15 mL), dried over anhydrous Na₂SO₄ and filtered. The filtrate was concentrated under reduced pressure to afford 5-((3,5-dimethoxyphenyl)ethynyl)-7-((5R)-5-(methoxymethyl)pyrrolidin-3-yl)pyrrolo[2,1-f][1,2,4]triazin-4-amine (30 mg). The crude product was used in the next step directly without further purification. MS ESI calculated for C₂₂H₂₅N₅O₃ [M+H]⁺, 408.20, found 408.20.

### Preparation 10: 1-(4-[4-Amino-5-[2-(3,5-dimethoxyphenyl)ethynyl]pyrrolo[2,1-f][1,2,4]triazin-7-yl]-2-(methoxymethyl)pyrrolidin-1-yl)prop-2-en-1-one

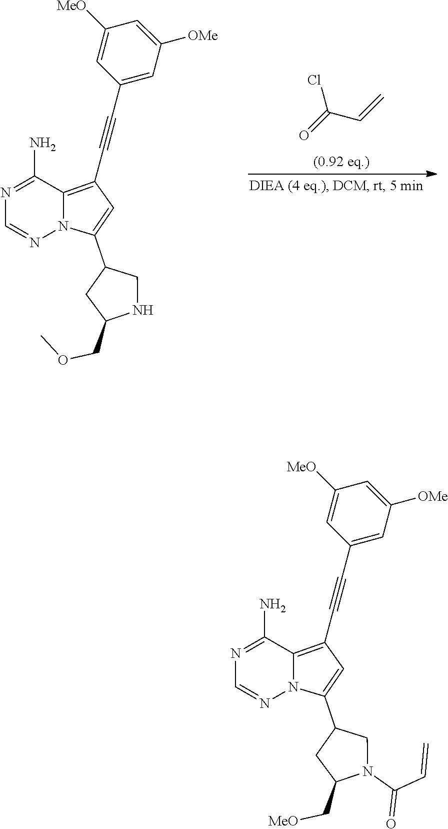

To a stirred solution of 5-[2-(3,5-dimethoxyphenyl)ethynyl]-7-[5-(methoxymethyl)pyrrolidin-3-yl]pyrrolo[2,1-f][1,2,4]triazin-4-amine (30.00 mg, 0.074 mmol), DIEA (38.06 mg, 0.29 mmol) in DCM (0.90 mL) was added acryloyl chloride (0.27 mL, 0.068 mmol) at 0° C. The reaction mixture was stirred for 5 min at 0° C. The resulting mixture was quenched with MeOH at room temperature and concentrated under reduced pressure. The residue was purified by Prep-HPLC with the following conditions: Column: XBridge C18 OBD Prep Column, 100 Å, 10 μm, 19 mm×250 mm; Mobile Phase A: Water (10 mmoL/L NH₄HCO₃), Mobile Phase B: ACN; Flow rate: 20 mL/min; Gradient: 20 B to 70 B in 4.8 min. The fractions contained desired product were combined and concentrated to afford 1-(4-[4-amino-5-[2-(3,5-dimethoxyphenyl)ethynyl]pyrrolo[2,1-f][1,2,4]triazin-7-yl]-2-(methoxymethyl)pyrrolidin-1-yl)prop-2-en-1-one (8.6 mg, 25%) as a white solid. MS ESI calculated for C₂₅H₂₇N₅O₄ [M+H]⁺, 462.21, found 462.25. H-NMR (400 MHz, d₆-DMSO) δ 7.99 (s, 1H), 6.85-6.56 (m, 7H), 6.18-6.14 (m, 1H), 5.71-5.68 (m, 1H), 4.42-3.97 (m, 3H), 3.78-3.34 (m, 12H), 2.32-2.20 (m, 2H).

### Preparation 11: 1-((2R,4S)-4-(4-amino-5-((3,5-dimethoxyphenyl)ethynyl)pyrrolo[2,1-f][1,2,4]triazin-7-yl)-2-(methoxymethyl)pyrrolidin-1-yl)prop-2-en-1-one and 1-((2R,4R)-4-(4-amino-5-((3,5-dimethoxyphenyl)ethynyl)pyrrolo[2,1-f][1,2,4]triazin-7-yl)-2-(methoxymethyl)pyrrolidin-1-yl)prop-2-en-1-one

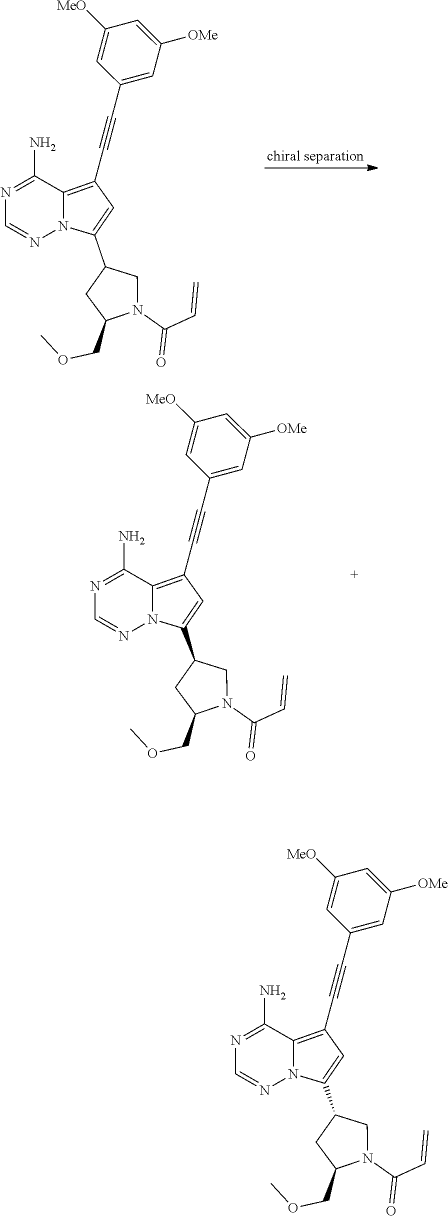

1-(4-[4-Amino-5-[2-(3,5-dimethoxyphenyl)ethynyl]pyrrolo[2,1-f][1,2,4]triazin-7-yl]-2-(methoxymethyl)pyrrolidin-1-yl)prop-2-en-1-one (0.12 g, 0.2 mmol) was purified by Prep-Chiral-HPLC with the following conditions Column: CHIRALPAK IA, 2×25 cm, 5 um; Mobile Phase A: MTBE (10 mM NH₃-MeOH)-HPLC, Mobile Phase B: EtOH-HPLC; Flow rate: 13 mL/min; Gradient: 50 B to 50 B in 22 min; 220/254 nm. The fractions contained desired product were concentrated to give the two enantiomers: 8 mg (6%) of the first eluted isomer Example 1 (RT=10.24 min, ee >98%, off-white solid) and 78 mg (64%) of the second eluted isomer Example 2 (RT=15.60 min, ee >98%, off-white solid).

¹H-NMR (400 MHz, d₆-DMSO) of the first eluted isomer, Example 1: δ 7.99 (s, 1H), 6.86-6.55 (m, 7H), 6.18-6.14 (m, 1H), 5.72-5.68 (m, 1H), 4.42-3.97 (m, 3H), 3.78-3.34 (m, 12H), 2.32-2.20 (m, 2H). MS ESI calculated for C₂₅H₂₇N₅O₄ [M+H]⁺, 462.21, found 462.25.

H-NMR (400 MHz, d₆-DMSO) of the second eluted isomer, Example 2: δ 8.0 (s, 1H), 6.88-6.55 (m, 7H), 6.19-6.14 (m, 1H), 5.71-5.67 (m, 1H), 4.42-3.98 (m, 3H), 3.78-3.33 (m, 12H), 2.32-2.21 (m, 2H). MS ESI calculated for C₂₅H₂₇N₅O₄ [M+H]⁺, 462.21, found 462.25.

### Example 3 and 4: 1-1(2R,4R)-4-[4-amino-5-[2-(2,6-difluoro-3,5-dimethoxyphenyl)ethynyl]pyrrolo[2,1-f][1,2,4]triazin-7-yl]-2-(methoxymethyl)pyrrolidin-1-yl]prop-2-en-1-one

### And

### 1-[(2R,4S)-4-[4-amino-5-[2-(2,6-difluoro-3,5-dimethoxyphenyl)ethynyl]pyrrolo[2,1-f][1,2,4]triazin-7-yl]-2-(methoxymethyl)pyrrolidin-1-yl]prop-2-en-1-one

### Preparation 1: 3-Ethynyl-2,4-difluoro-1,5-dimethoxybenzene and 1-ethynyl-2-fluoro-3,5-dimethoxybenzene

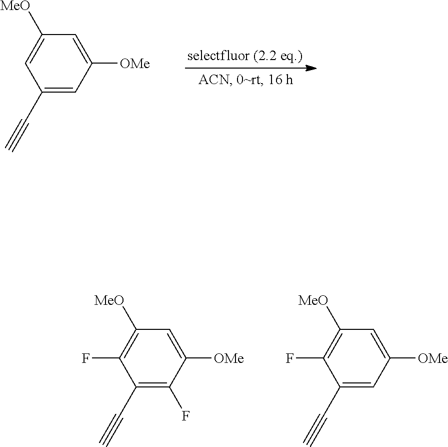

To a stirred solution of 1-ethynyl-3,5-dimethoxybenzene (275.0 g, 1.70 mol) in ACN (11.00 L) was added Selectfluor (1.20 kg, 3.39 mol) in portions at 0° C. under nitrogen atmosphere. The reaction mixture was stirred overnight at room temperature under nitrogen atmosphere. The resulting mixture was concentrated under reduced pressure. The resulting mixture was diluted with water (3 L), and extracted with EtOAc (3×4 L). The combined organic layers were washed with brine (2×2 L), dried over anhydrous Na₂SO₄. After filtration, the filtrate was concentrated under reduced pressure. The residue was purified by silica gel column chromatography, eluted with PE/EtOAc (100:1). The fractions contained desired product were combined and concentrated to afford 3-ethynyl-2, 4-difluoro-1,5-dimethoxybenzene (51.46 g, 17%) as a white solid and 1-ethynyl-2-fluoro-3,5-dimethoxybenzene (8.88 g, 3.2%) as a yellow solid. 3-ethynyl-2, 4-difluoro-1,5-dimethoxybenzene: ¹H-NMR (400 MHz, d₆-DMSO) δ 6.66 (t, J=8.0 Hz, 1H), 3.88 (s, 6H), 3.52 (s, 1H). 1-ethynyl-2-fluoro-3,5-dimethoxybenzene: H-NMR (400 MHz, d₆-DMSO) δ 6.55-6.48 (m, 2H), 3.86 (s, 3H), 3.77 (s, 3H), 3.29 (s, 1H).

### Preparation 2: 5-iodo-7-[(5R)-5-(methoxymethyl)pyrrolidin-3-yl]pyrrolo[2,1-f][1,2,4]triazin-4-amine

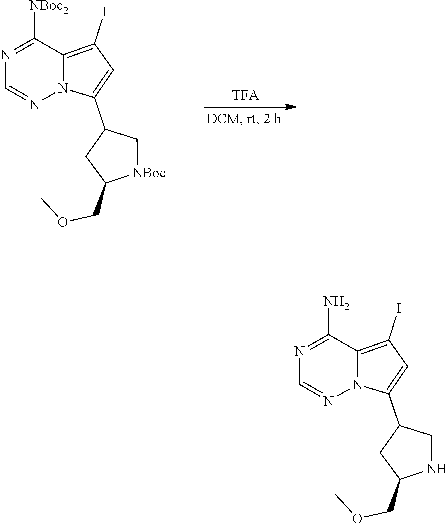

To a stirred solution of tert-butyl (2R)-4-[4-[bis(tert-butoxycarbonyl)amino]-5-iodopyrrolo[2,1-f][1,2,4]triazin-7-yl]-2-(methoxymethyl)pyrrolidine-1-carboxylate (4.27 g, 6.34 mmol) in DCM (32.00 mL) were added TFA (8.00 mL, 0.07 mmol) dropwise at room temperature. The reaction mixture was stirred for 2 h at room temperature. The resulting mixture was concentrated under reduced pressure. The residue was purified by reverse flash chromatography with the following conditions: column, C18 silica gel; mobile phase, A: NH₄HCO₃ in water and B: ACN, 0% to 40% A gradient in 30 min; detector, UV 254 nm. The fractions contained desired product were combined and concentrated to afford 5-iodo-7-[(5R)-5-(methoxymethyl)pyrrolidin-3-yl]pyrrolo[2,1-f][1,2,4]triazin-4-amine (2.7 g, 97%) as a white solid. MS ESI calculated for C₁₂H₁₆IN₅O [M+H]⁺, 374.04, found 373.95.

### Preparation 3: 1-[(2R)-4-[4-amino-5-iodopyrrolo[2,1-f][1,2,4]triazin-7-yl]-2-(methoxymethyl)pyrrolidin-1-yl]prop-2-en-1-one

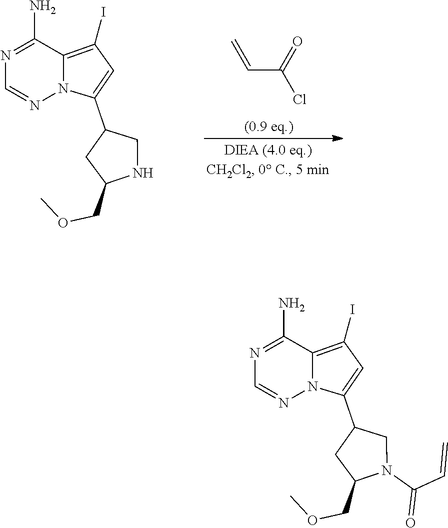

To a stirred solution of 5-iodo-7-[(5R)-5-(methoxymethyl)pyrrolidin-3-yl]pyrrolo[2,1-f][1,2,4]triazin-4-amine (1.1 g, 2.95 mmol) and DIEA (1.52 g, 11.79 mmol) in DCM (29.4 mL) was added acryloyl chloride (0.24 g, 2.65 mmol) in DCM (10.9 mL) slowly at 0° C. The reaction mixture was stirred for 5 min at 0° C. The resulting mixture was quenched with water (50 mL) at room temperature. The resulting mixture was extracted with DCM (3×40 mL). The combined organic layers were washed with brine (80 mL), dried over anhydrous Na₂SO₄ and filtered. The filtrate was concentrated under reduced pressure. The residue was purified by silica gel column chromatography, eluted with DCM/MeOH (10:1). The fractions contained desired product were combined and concentrated to afford 1-[(2R)-4-[4-amino-5-iodopyrrolo[2,1-f][1,2,4]triazin-7-yl]-2-(methoxymethyl)pyrrolidin-1-yl]prop-2-en-1-one (1.19 g, 93%) as an off-white solid. MS ESI calculated for C₁₅H₁₈IN₅O₂ [M+H]⁺, 428.05, found 428.00.

### Preparation 4: 1-[(2R)-4-[4-amino-5-iodopyrrolo[2,1-f][1,2,4]triazin-7-yl] (methoxymethyl)pyrrolidin-1-yl]prop-2-en-1-one

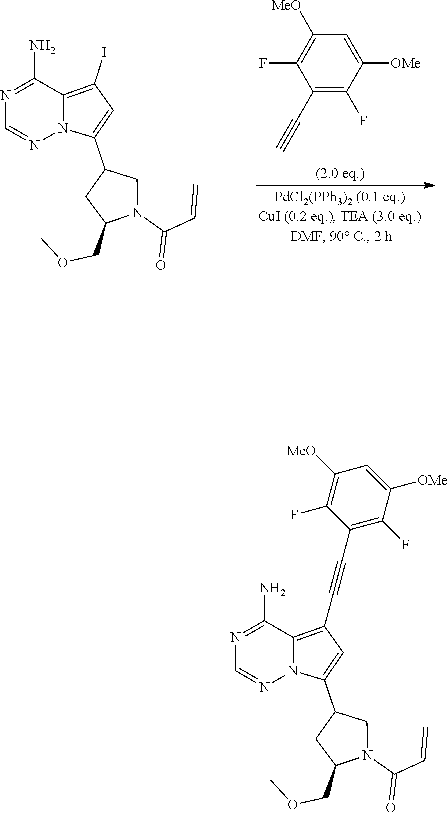 [SMILES: C=CC(=O)N1CC(c2cc(I)c3c(N)ncnn23)C[C@@H]1COC ; C#Cc1c(F)c(OC)cc(OC)c1F ; C=CC(=O)N1CC(c2cc(C#Cc3c(F)c(OC)cc(OC)c3F)c3c(N)ncnn23)C[C@@H]1COC]

To a stirred mixture of 1-[(2R)-4-[4-amino-5-iodopyrrolo[2,1-f][1,2,4]triazin-7-yl]-2-(methoxymethyl)pyrrolidin-1-yl]prop-2-en-1-one (0.22 g, 0.503 mmol), 3-ethynyl-2,4-difluoro-1,5-dimethoxybenzene (0.20 g, 1.01 mmol) and CuI (19.17 mg, 0.10 mmol) in DMF (5.00 mL) were added TEA (0.15 g, 1.51 mmol) and Pd(PPh₃)₂Cl₂ (35.32 mg, 0.05 mmol) under argon atmosphere. The resulting mixture was degassed with argon for three times and stirred for 2 h at 90° C. The resulting mixture was diluted with water (20 mL) and extracted with EtOAc (3×20 mL). The combined organic layers was washed with brine (150 mL), dried over anhydrous Na₂SO₄ and filtered. The filtrate was concentrated under reduced pressure. The residue was purified by silica gel column chromatography, eluted with CH₂Cl₂/MeOH (10:1). The crude product was further purified by Prep-HPLC with the following conditions Column: XBridge BEH C18 OBD Prep Column, 5 μm, 19 mm×250 mm; Mobile Phase A: Water (10 mmol/L NH₄HCO₃), Mobile Phase B: ACN; Flow rate: 20 mL/min; Gradient: 30 B to 70 B in 5 min. The fractions contained desired product were combined and concentrated to afford 1-[(2R)-4-[4-amino-5-[2-(2,6-difluoro-3,5-dimethoxyphenyl)ethynyl]pyrrolo[2,1-f][1,2,4]triazin-7-yl]-2-(methoxymethyl)pyrrolidin-1-yl]prop-2-en-1-one (85 mg, 33%) as an off-white solid. MS ESI calculated for C₂₅H₂₅F₂N₅O₄ [M+H]⁺, 498.19, found 498.15.

### Preparation 5: 1-[(2R,4R)-4-[4-amino-5-[2-(2,6-difluoro-3,5-dimethoxyphenyl)ethynyl]pyrrolo[2,1-f][1,2,4]triazin-7-yl]-2-(methoxymethyl)pyrrolidin-1-yl]prop-2-en-1-one and 1-[(2R,4S)-4-[4-amino-5-[2-(2,6-difluoro-3,5-dimethoxyphenyl)ethynyl]pyrrolo[2,1-f][1,2,4]triazin-7-yl]-2-(methoxymethyl)pyrrolidin-1-yl]prop-2-en-1-one

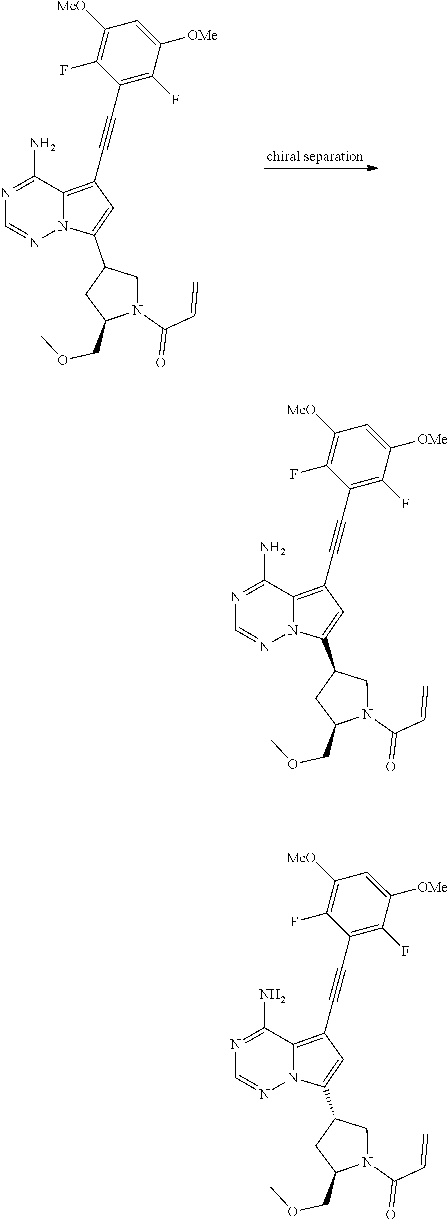 [SMILES: C=CC(=O)N1CC(c2cc(C#Cc3c(F)c(OC)cc(OC)c3F)c3c(N)ncnn23)C[C@@H]1COC ; C=CC(=O)N1C[C@H](c2cc(C#Cc3c(F)c(OC)cc(OC)c3F)c3c(N)ncnn23)C[C@@H]1COC ; C=CC(=O)N1C[C@@H](c2cc(C#Cc3c(F)c(OC)cc(OC)c3F)c3c(N)ncnn23)C[C@@H]1COC]

1-[(2R)-4-[4-amino-5-[2-(2,6-difluoro-3,5-dimethoxyphenyl)ethynyl]pyrrolo[2,1-f][1,2,4]triazin-7-yl]-2-(methoxymethyl)pyrrolidin-1-yl]prop-2-en-1-one (0.10 g, 0.16 mmol) was purified by Prep-Chiral-HPLC with the following conditions Column: CHIRALPAK IA, 2×25 cm, 5 um; Mobile Phase A: MTBE (10 mM NH₃-MeOH)-HPLC, Mobile Phase B: EtOH-HPLC; Flow rate: 13 mL/min; Gradient: 50 B to 50 B in 22 min; 220/254 nm. The fractions contained desired product were concentrated to give the two enantiomers: 7.8 mg (8%) of the first eluted isomer Example 3 (RT=12.7 min, ee >98%, off-white solid) and 65.2 mg (65%) of the second eluted isomer Example 4 (RT=18.3 min, ee >98%, off-white solid).

¹H-NMR (400 MHz, d₆-DMSO) of the first eluted isomer, Example 3: δ 7.99 (s, 1H), 7.29-7.19 (m, 2H), 7.07 (t, J=11.2 Hz, 1H), 6.90 (s, 1H), 6.63-6.60 (m, 1H), 6.18-6.12 (m, 1H), 5.69-5.65 (m, 1H), 4.33-4.31 (m, 2H), 3.92 (s, 6H), 3.88-3.41 (m, 4H), 3.29 (s, 3H), 2.73-2.50 (m, 1H), 2.13-2.08 (m, 1H). MS ESI calculated for C₂₅H₂₅F₂N₅O₄ [M+H]⁺, 498.19, found 498.10. H-NMR (400 MHz, d₆-DMSO) of the second eluted isomer, Example 4: δ 8.00 (s, 1H), 7.28-7.15 (m, 2H), 7.07 (t, J=11.2 Hz, 1H), 6.85 (s, 1H), 6.63-6.60 (m, 1H), 6.18-6.12 (m, 1H), 5.67-5.63 (m, 1H), 4.38-4.34 (m, 1H), 4.06-3.88 (m, 8H), 3.70-3.41 (m, 3H), 3.34 (s, 3H), 2.50-2.28 (m, 2H). MS ESI calculated for C₂₅H₂₅F₂N₅O₄ [M+H]⁺, 498.19, found 498.10.

### Example 5 and 6: 1-[(2R,4R)-4-[4-amino-5-[2-(2-fluoro-3,5-dimethoxyphenyl)ethynyl]pyrrolo[2,1-f][1,2,4]triazin-7-yl]-2-(methoxymethyl)pyrrolidin-1-yl]prop-2-en-1-one

### And

### 1-[(2R,4S)-4-[4-amino-5-[2-(2-fluoro-3,5-dimethoxyphenyl)ethynyl]pyrrolo[2,1-f][1,2,4]triazin-7-yl]-2-(methoxymethyl)pyrrolidin-1-yl]prop-2-en-1-one

### Preparation 1: 1-[(2R)-4-[4-amino-5-[2-(2-fluoro-3,5-dimethoxyphenyl)ethynyl]pyrrolo[2,1-f][1,2,4]triazin-7-yl]-2-(methoxymethyl)pyrrolidin-1-yl]prop-2-en-1-one

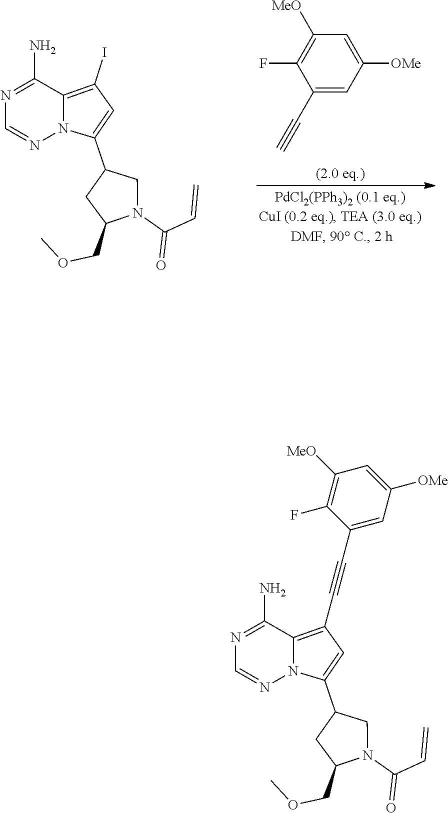 [SMILES: C=CC(=O)N1CC(c2cc(I)c3c(N)ncnn23)C[C@@H]1COC ; C#Cc1cc(OC)cc(OC)c1F ; C=CC(=O)N1CC(c2cc(C#Cc3cc(OC)cc(OC)c3F)c3c(N)ncnn23)C[C@@H]1COC]

To a stirred mixture of 1-[(2R)-4-[4-amino-5-iodopyrrolo[2,1-f][1,2,4]triazin-7-yl]-2-(methoxymethyl)pyrrolidin-1-yl]prop-2-en-1-one (0.22 g, 0.503 mmol), 1-ethynyl-2-fluoro-3,5-dimethoxybenzene (0.18 g, 1.01 mmol) and CuI (19.17 mg, 0.10 mmol) in DMF (5.00 mL) were added TEA (0.15 g, 1.51 mmol) and Pd(PPh₃)₂Cl₂ (35.32 mg, 0.05 mmol) under argon atmosphere. The resulting mixture was degassed with argon for three times and stirred for 2 h at 90° C. The resulting mixture was diluted with water (20 mL) and extracted with EtOAc (3×20 mL). The combined organic layer was washed with brine (150 mL), dried over anhydrous Na₂SO₄ and filtered. The filtrate was concentrated under reduced pressure. The residue was purified by silica gel column chromatography, eluted with CH₂Cl₂/MeOH (10:1). The crude product was further purified by Prep-HPLC with the following conditions Column: XBridge BEH C18 OBD Prep Column, 5 μm, 19 mm×250 mm; Mobile Phase A: Water (10 mmol/L NH₄HCO₃), Mobile Phase B: ACN; Flow rate: 20 mL/min; Gradient: 35 B to 75 B in 5 min. The fractions contained desired product were combined and concentrated to afford 1-[(2R)-4-[4-amino-5-[2-(2-fluoro-3,5-dimethoxyphenyl)ethynyl]pyrrolo[2,1-f][1,2,4]triazin-7-yl]-2-(methoxymethyl)pyrrolidin-1-yl]prop-2-en-1-one (0.11 g, 44%) as an off-white solid. MS ESI calculated for C₂₅H₂₆FN₅O₄ [M+H]⁺, 480.20, found 480.20.

### Preparation 2: 1-[(2R,4R)-4-[4-amino-5-[2-(2-fluoro-3,5-dimethoxyphenyl)ethynyl]pyrrolo[2,1-f][1,2,4]triazin-7-yl]-2-(methoxymethyl)pyrrolidin-1-yl]prop-2-en-1-one and 1-[(2R,4S)-4-[4-amino-5-[2-(2-fluoro-3,5-dimethoxyphenyl)ethynyl]pyrrolo[2,1-f][1,2,4]triazin-7-yl]-2-(methoxymethyl)pyrrolidin-1-yl]prop-2-en-1-one

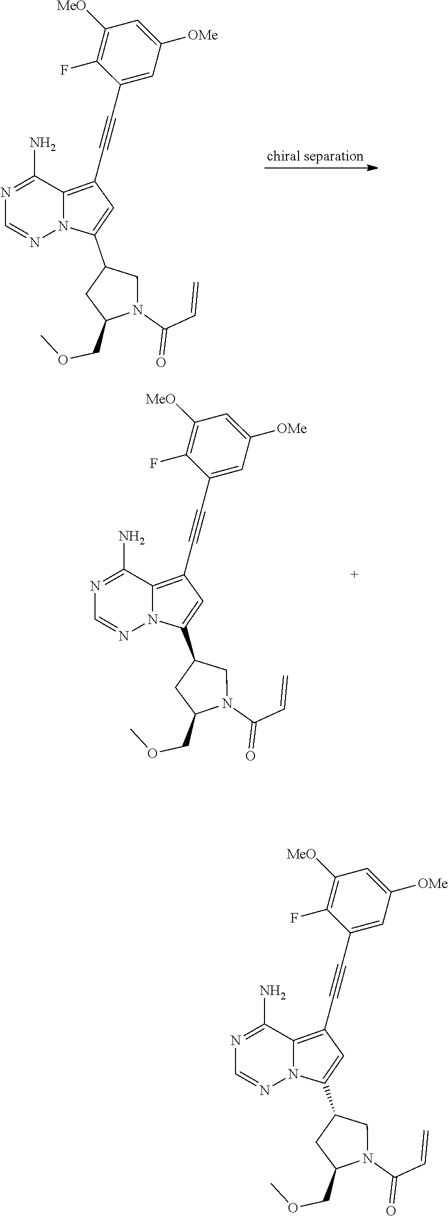

1-[(2R)-4-[4-amino-5-[2-(2-fluoro-3,5-dimethoxyphenyl)ethynyl]pyrrolo[2,1-f][1,2,4]triazin yl]-2-(methoxymethyl)pyrrolidin-1-yl]prop-2-en-1-one (0.11 g, 0.23 mmol) was purified by Prep-Chiral-HPLC with the following conditions Column: CHIRALPAK IA, 2×25 cm, 5 um; Mobile Phase A: MTBE (2 mM NH₃-MeOH)-HPLC, Mobile Phase B: EtOH-HPLC; Flow rate: 16 mL/min; Gradient: 50 B to 50 B in 21 min; 254/220 nm. The fractions contained desired product were concentrated to give the two enantiomers: 9.7 mg (8.8%) of the first eluted isomer Example 5 (RT=11.2 min, ee >98%, off-white solid) and 83.1 mg (76%) of the second eluted isomer Example 6 (RT=17.8 min, ee >98%, off-white solid). ¹H-NMR (400 MHz, d₆-DMSO) of the first eluted isomer, Example 5: δ 7.97 (s, 1H), 7.24 (s, 1H), 6.85 (s, 1H), 6.81-6.80 (m, 1H), 6.78-6.67 (m, 2H), 6.18-6.12 (m, 1H), 5.72-5.68 (m, 1H), 4.33-4.31 (m, 2H), 3.89 (s, 3H), 3.81 (s, 3H), 3.74-3.45 (m, 4H), 3.29 (s, 3H), 2.65-2.50 (m, 2H), 2.22-2.05 (m, 1H). MS ESI calculated for C₂₅H₂₆FN₅O₄ [M+H]⁺, 480.20, found 480.15.

¹H-NMR (400 MHz, d₆-DMSO) of the second eluted isomer, Example 6: δ 7.98 (s, 1H), 7.23 (s, 1H), 6.85 (s, 1H), 6.80-6.77 (m, 2H), 6.69-6.58 (m, 2H), 6.18-6.12 (m, 1H), 5.67-5.62 (m, 1H), 4.38-4.37 (m, 1H), 4.05-4.01 (m, 2H), 3.89 (s, 3H), 3.85 (s, 3H), 3.70-3.34 (m, 3H), 3.34 (s, 3H), 2.52-2.37 (m, 2H). MS ESI calculated for C₂₅H₂₆FN₅O₄ [M+H]⁺, 480.20, found 480.15.

II. Biological Evaluation

### Example 1: FGFR WT and FGFR2 V565F Mutation Kinase Protocol

Biochemical assays were run at Reaction Biology (Reaction Biology Corporation, 1 Great Valley Parkway, Suite 2, Malvern, Pa. 19355, USA). The Reaction Biology (http://www.reactionbiology.com) HotSpot assay platform was used to measure kinase/inhibitor interactions as described previously (Anastassiadis et al., 2011). In brief, for each reaction, kinase and substrate were mixed in a buffer containing 20 mM HEPES (pH 7.5), 10 mM MgCl₂, 1 mM EGTA, 0.02% Brij35, 0.02 mg/mL BSA, 0.1 mM Na₃VO₄, 2 mM DTT, and 1% DMSO. All compounds were in powder form and freshly solubilized in DMSO for assays. Compounds were then added to each reaction mixture via acoustic dispensing using an ECHO 550 nanoliter dispenser. For Human FGFR2 wildtype testing, a peptide substrate, poly[Glu:Tyr] (4:1) was used to promote the reaction at a concentration of 0.2 mg/ml, with a final ATP concentration as determined by Reaction Biology. For Human FGFR2 (V565F) testing, a peptide substrate, poly[Glu:Tyr] (4:1) was used to promote the reaction at a concentration of 0.2 mg/ml, with a final ATP concentration as determined by Reaction Biology. Compounds were tested in 10-dose IC50 mode with a 3-fold serial dilution. After a 20-min incubation, ATP (Sigma-Aldrich, St. Louis, Mo. 63178) and [g33P] ATP (specific activity 10 microCi/microliter) purchased at PerkinElmer (Boston, Mass., 02118 Cat #BLU 003H250UC) were added at a final total concentration of 10 mM. Reactions were carried out at room temperature for 2 hr and spotted onto P81 ion exchange cellulose chromatography paper (Reaction Biology). Filter paper was washed in 0.75% phosphoric acid to remove unincorporated ATP. The percent remaining kinase activity relative to a vehicle-containing (DMSO) kinase reaction was calculated for each kinase/inhibitor pair. IC50 values were calculated using Prism 5 (GraphPad).

TABLE 3

| | FGFR2 (WT) | FGFR2 (V565F) |
|---|---|---|
| | Km | 100 uM ATP |
| Compound | IC₅₀ (nM) | IC₅₀ (nM) |
| Chemical Synthesis | A | B |
| Example 2 | | |
| Chemical Synthesis | A | A |
| Example 4 | | |
| Chemical Synthesis | A | B |
| Example 6 | | |

Note:
Biochemical assay IC₅₀ data are designated within the following ranges:
A: ≤0.010 μM
B: >0.010 μM to ≤0.10 μM
C: >0.10 μM to ≤1.0 μM
D: >1.0 μM to ≤10 μM

SNU-16 Cell-Based Viability Assay.

For determining the covalent nature of compounds, a simple cell-based assay was employed. SNU-16 cells were obtained from ATCC (Cat #CRL-5974; American Type Culture Collection; Manassas, Va. 20110) for this assay. SNU-16 is a cultured line derived in 1987 by J. Park and associates from ascites of a patient with poorly differentiated gastric carcinoma of the stomach, and cells were collected from the patient prior to chemotherapy. The cells express the surface glycoproteins carcinoembryonic antigen (CEA) and TAG-72. No evidence of amplification or rearrangements has been noted in the N-myc, L-myc, myb and EGF receptor genes, although the c-myc proto-oncogene is amplified. There was no expression of the following genes: N-myc, L-myc, c-cis, IGF-2, or gastrin releasing peptide. These cells are known to have an FGFR2 amplification and are therefore relevant for the compound testing at hand (1). For this assay, cells were plated on poly-D-lysine to maintain attachment. Cells were either exposed to compound for 2 hours or 72 hours to test the ability of the compound to stay on target after binding. Briefly, 80 μL cell solution containing 5000 SNU-16 cells was plated per well into 96-well Poly-D-Lysine Cellware plate and incubated in 37° C. incubator overnight to facilitate adherence. Compounds were solubilized fresh from powder as dimethylsulfoxide stocks (DMSO), and plated as half log, 9 point dilutions in DMSO and then plated into cell media containing RPMI1640+10% FBS for ease of transfer without disrupting cells. Total volume in cells was 100 uL. For cells exposed to compound for two hours, cells were incubated in 37° C. incubator for 2 hours, upon which time plates were removed and cells were centrifuged at 1500 rpm for 5 min, followed by removal of all supernatant, and the re-dispense 100 μL of RPMI1640+10% FBS per well. This was repeated twice. Plates were put back into the incubator for 72 hours. For cells exposed to compound for 72 hours, plates were removed from the incubator for the time of washing but not disturbed, and then placed back in the incubator without washing. Assay plates were read for total ATP by luminescent CTG signal at the end of the 72 hour incubation by adding 100 μL of CTG (Cell TiterGlo: Cat #G9241; Promega Corporation, Madison, Wis., 53711) per well, followed by gently agitating the contents for 2 min on the plate shaker, and incubating the plates for 10 min at room temperature in dark. Luminescent signal was read on as Envision plate reader, according to manufacturer's suggestions. The inhibition activity was calculated using Xlfit (v5.3.1.3), equation 201: Y=Bottom+(Top−Bottom)/(1+10\hat{ }((Log IC50−X)*HillSlope)).
1) Zhao W M, Wang L, Park H, Chim S, Tanphanich M, Yashiro M, et al. Monoclonal antibodies to fibroblast growth factor receptor 2 effectively inhibit growth of gastric tumor xenografts. Clin Cancer Res 2010; 16:5750-8.

Exemplary compounds are presented in Table 4.

TABLE 4

| | SNU-16 | SNU-16 |
|---|---|---|
| | IC₅₀ (nM) | IC₅₀ (nM) |
| Compound | 72 h | 2 h |
| Chemical Synthesis | B | D |
| Example 1 | | |
| Chemical Synthesis | A | A |
| Example 2 | | |
| Chemical Synthesis | C | D |
| Example 3 | | |
| Chemical Synthesis | A | A |
| Example 4 | | |
| Chemical Synthesis | C | D |
| Example 5 | | |
| Chemical Synthesis | A | A |
| Example 6 | | |

Note:
Biochemical assay IC₅₀ data are designated within the following ranges:
A: ≤0.010 μM
B: >0.010 μM to ≤0.10 μM
C: >0.10 μM to ≤1.0 μM
D: >1.0 μM to ≤10 μM

III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt thereof, and is formulated as a solution in sesame oil at a concentration of 50 mg−eq/mL.

The examples and embodiments described herein are for illustrative purposes only and various modifications or changes suggested to persons skilled in the art are to be included within the spirit and purview of this application and scope of the appended claims.

1. A compound, or pharmaceutically acceptable salt or solvate thereof, having the structure of Formula (I): 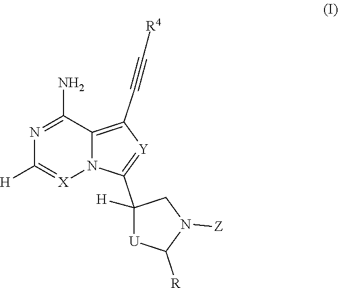
wherein,
X is C—H or N;
Y is C—H or N;
Z is selected from a group having the structure: 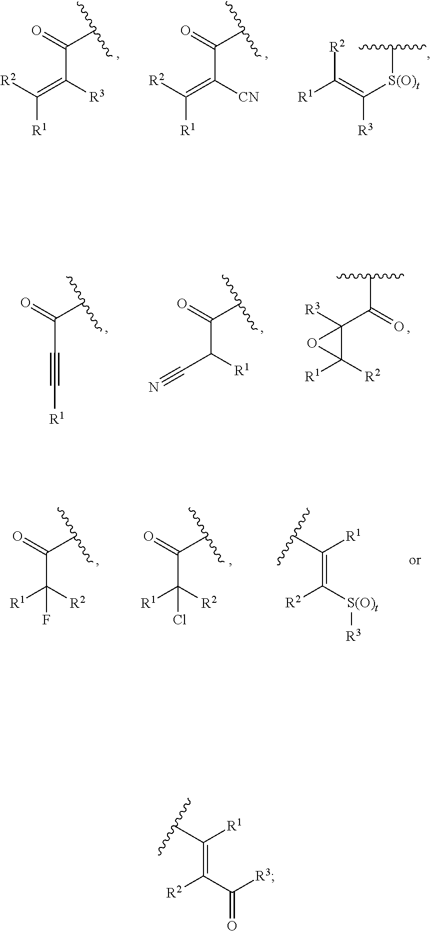
t is 1 or 2;
R¹, R², and R³ are each independently selected from hydrogen, optionally substituted C1-C4 alkyl, or optional substituted heterocyclylalkyl;
R⁴ is an optionally substituted C6 aryl;
U is —CH₂—, or a bond;
R is selected from optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂; and
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl.

2. The compound of claim 1, or a pharmaceutically acceptable salt or solvate thereof, wherein X is C—H, and Y is C—H.

3. The compound of claim 1, or a pharmaceutically acceptable salt or solvate thereof, wherein X is C—H, and Y is N.

4. The compound of claim 1, or a pharmaceutically acceptable salt or solvate thereof, wherein X is N, and Y is C—H.

5. The compound of claim 1, or a pharmaceutically acceptable salt or solvate thereof, wherein X is N, and Y is N.

6. The compound of claim 1, or a pharmaceutically acceptable salt or solvate thereof, wherein Z is

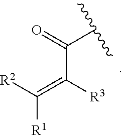

7. (canceled)

8. (canceled)

9. The compound of claim 6, or pharmaceutically acceptable salt or solvate thereof, wherein R² and R³ are hydrogen.

10. The compound of claim 6, or pharmaceutically acceptable salt or solvate thereof, wherein R¹ is hydrogen.

11. (canceled)

12. (canceled)

13. (canceled)

14. (canceled)

15. (canceled)

16. (canceled)

17. The compound of claim 1, or a pharmaceutically acceptable salt or solvate thereof, wherein U is —CH₂—.

18. The compound of claim 1, or a pharmaceutically acceptable salt or solvate thereof, wherein R⁴ is an unsubstituted phenyl.

19. The compound of claim 1, or a pharmaceutically acceptable salt or solvate thereof, wherein R⁴ is a substituted phenyl.

20. The compound of claim 1, or a pharmaceutically acceptable salt or solvate thereof, wherein R⁴ is a substituted phenyl having a hydrogen at the 4-position.

21. (canceled)

22. (canceled)

23. (canceled)

24. (canceled)

25. The compound of claim 19, or a pharmaceutically acceptable salt or solvate thereof, wherein the substituted phenyl is substituted with at least one substituent selected from halogen, —CN, optionally substituted C1-C4 alkyl, or optionally substituted C1-C3 alkoxy.

26. The compound of claim 1, or a pharmaceutically acceptable salt or solvate thereof, wherein R is optionally substituted C1-C6 alkyl.

27. The compound of claim 1, or a pharmaceutically acceptable salt or solvate thereof, wherein R is optionally substituted C3-C7 carbocyclyl.

28. The compound of claim 1, or a pharmaceutically acceptable salt or solvate thereof, wherein R is optionally substituted C3-C7 carbocyclylalkyl.

29. The compound of claim 1, or a pharmaceutically acceptable salt or solvate thereof, wherein R is optionally substituted C3-C7 heterocyclyl.

30. The compound of claim 1, or a pharmaceutically acceptable salt or solvate thereof, wherein R is optionally substituted C3-C7 heterocyclylalkyl.

31. The compound of claim 1, or a pharmaceutically acceptable salt or solvate thereof, wherein R is optionally substituted C2-C7 alkenyl.

32. The compound of claim 1, or a pharmaceutically acceptable salt or solvate thereof, wherein R is —CO₂R⁵.

33. The compound of claim 1, or a pharmaceutically acceptable salt or solvate thereof, wherein R is —CONHR⁵.

34. The compound of claim 1, or a pharmaceutically acceptable salt or solvate thereof, wherein R is —CON(R⁵)₂.

35. A pharmaceutical composition comprising a compound of Formula (I), or pharmaceutically acceptable salt or solvate thereof, as described in claim 1, and a pharmaceutically acceptable excipient.

36. (canceled)

37. (canceled)

38. (canceled)

39. (canceled)

40. A method of treating cancer in a patient in need thereof comprising administering to the patient a compound of Formula (I) as described in claim 1, or pharmaceutically acceptable salt or solvate thereof.

41. (canceled)
